SEQUENCE LISTING (55 sequences)

SEQ ID NO: 1 - DNA, 15 bp, synthetic construct
  source 1..15: mol_type = other DNA
ggatcttatcatggt

SEQ ID NO: 2 - DNA, 15 bp, synthetic construct
  source 1..15: mol_type = other DNA
ggatcctatcatggt

SEQ ID NO: 3 - DNA, 15 bp, synthetic construct
  source 1..15: mol_type = other DNA
ggatcatatcatggt

SEQ ID NO: 4 - DNA, 15 bp, synthetic construct
  source 1..15: mol_type = other DNA
ggatcgtatcatggt

SEQ ID NO: 5 - DNA, 15 bp, synthetic construct
  source 1..15: mol_type = other DNA
ggatcctatcatggt

SEQ ID NO: 6 - DNA, 15 bp, synthetic construct
  source 1..15: mol_type = other DNA
ggatcttatcatggt

SEQ ID NO: 7 - protein, 5 aa, synthetic construct
GSYHG

SEQ ID NO: 8 - protein, 34 aa, Xanthomonas oryzae
  VARIANT 12: note = X can be any amino acid
  VARIANT 13: note = X can be any amino acid
LTPDQVVAIASXXGGKQALETVQRLLPVLCQDHG

SEQ ID NO: 9 - protein, 34 aa, Xanthomonas oryzae
  VARIANT 4: note = B can be D E or A
  VARIANT 12: note = X can be any amino acid
  VARIANT 13: note = X can be any amino acid
  VARIANT 32: note = J can be D or A
LTPBQVVAIASXXGGKQALETVQRLLPVLCQJHG

SEQ ID NO: 10 - protein, 34 aa, Xanthomonas oryzae
  VARIANT 12: note = X can be any amino acid
  VARIANT 13: note = X can be any amino acid
  VARIANT 24: note = Z can be R or A
  VARIANT 20: note = B can be E or A
  VARIANT 22: note = J can be V or M
LTPDQVVAIASXXGGKQALBTJQZLLPVLCQDHG

SEQ ID NO: 11 - protein, 20 aa, Xanthomonas oryzae
  VARIANT 12: note = X can be any amino acid
  VARIANT 13: note = X can be any amino acid
LTPEQVVAIASXXGGRPALE

SEQ ID NO: 12 - protein, 21 aa, Xanthomonas oryzae
  VARIANT 12: note = X can be any amino acid
  VARIANT 13: note = X can be any amino acid
LTPDQVVAIASXXGGKQALES

SEQ ID NO: 13 - protein, 20 aa, Xanthomonas oryzae
  VARIANT 12: note = X can be any amino acid
  VARIANT 13: note = X can be any amino acid
LTPNQVVAIASXXGGKQALE

SEQ ID NO: 14 - protein, 7 aa, Simian virus 40
PKKKRKV

SEQ ID NO: 15 - protein, 20 aa, unidentified
AVKRPAATKKAGQAKKKKLD

SEQ ID NO: 16 - protein, 25 aa, unidentified
MSRRRKANPTKLSENAKKLAKEVEN

SEQ ID NO: 17 - protein, 9 aa, unidentified
PAAKRVKLD

SEQ ID NO: 18 - protein, 9 aa, unidentified
KLKIKRPVK

SEQ ID NO: 19 - protein, 36 aa, Arabidopsis thaliana
MFKQASRLLSRSVAAASSKSVTTRAFSTELPSTLDS

SEQ ID NO: 20 - protein, 95 aa, Oryza sativa
MAARRAASSLLSRGLIARPSAASSTGDSAILGAGSARGFLPGSLHRFSAAPAAAATAAATEEPIQPPVDVKYTKLLINGNFVDAASGKTFATVDP

SEQ ID NO: 21 - protein, 54 aa, Pisum sativum
MWRRLFTSPHLKTLSSSSLSRPRSAVAGIRCVDLSRHVATQSAASVKKRVEDVV

SEQ ID NO: 22 - DNA, 18 bp, synthetic construct
  source 1..18: mol_type = other DNA
tcgctattacgccagctg

SEQ ID NO: 23 - DNA, 25 bp, synthetic construct
  source 1..25: mol_type = other DNA
cacccttgacgcgattgcaaccacc

SEQ ID NO: 24 - DNA, 26 bp, synthetic construct
  source 1..26: mol_type = other DNA
aatcgcgtcaagggtggggggaaagc

SEQ ID NO: 25 - DNA, 20 bp, synthetic construct
  source 1..20: mol_type = other DNA
gattcattaatgcagctggc

SEQ ID NO: 26 - DNA, 21 bp, synthetic construct
  source 1..21: mol_type = other DNA
tatagggcgaattgggtaccg

SEQ ID NO: 27 - DNA, 33 bp, synthetic construct
  source 1..33: mol_type = other DNA
tctgccccccaccctggatgcaatagccactac

SEQ ID NO: 28 - DNA, 22 bp, synthetic construct
  source 1..22: mol_type = other DNA
agggtggggggcagacccgcac

SEQ ID NO: 29 - DNA, 17 bp, synthetic construct
  source 1..17: mol_type = other DNA
tgtggagctgagatctg

SEQ ID NO: 30 - DNA, 72 bp, Arabidopsis thaliana
  source 1..72: mol_type = genomic DNA
tcctactttgtggaatggatcccaaacaacgtcaagtccagtgtctgtgatattgcaccaaagggtttgaaa

SEQ ID NO: 31 - DNA, 72 bp, Arabidopsis thaliana
  source 1..72: mol_type = genomic DNA
tcctactttgttgaatggatcccaaacaatgtgaagtccagcgtctgcgacattccaccaactggaatcaaa

SEQ ID NO: 32 - DNA, 72 bp, Arabidopsis thaliana
  source 1..72: mol_type = genomic DNA
tcctactttgtggagtggatccccaacaacgtgaaatcaacagtctgtgatatcccacctactggtctgaag

SEQ ID NO: 33 - DNA, 72 bp, Arabidopsis thaliana
  source 1..72: mol_type = genomic DNA
tcctactttgtggagtggatccccaacaacgtgaaatcgacagtctgtgacatcccacctactggtctgaag

SEQ ID NO: 34 - DNA, 72 bp, Arabidopsis thaliana
  source 1..72: mol_type = genomic DNA
tcatactttgtggaatggatccccaacaacgtgaaatcaaccgtgtgtgacattccaccaacaggtctgaaa

SEQ ID NO: 35 - DNA, 72 bp, Arabidopsis thaliana
  source 1..72: mol_type = genomic DNA
tcttacttcgttgagtggatccccaacaacgtgaaatccagcgtttgtgatatcccaccaaaggggctcaaa

SEQ ID NO: 36 - DNA, 72 bp, Arabidopsis thaliana
  source 1..72: mol_type = genomic DNA
tcctactttgtggaatggatccctaacaacgtcaaatcaaccgtctgtgacattccccctactggtctgaag

SEQ ID NO: 37 - DNA, 72 bp, Arabidopsis thaliana
  source 1..72: mol_type = genomic DNA
tcctactttgtggaatggattccaaacaacgtgaagtcaagcgtctgtgacatagctccccgaggtctctca

SEQ ID NO: 38 - DNA, 72 bp, Arabidopsis thaliana
  source 1..72: mol_type = genomic DNA
tcatactttgttgaatggattccaaataacgtgaaatcaagcgtctgcgatatagctcccactggtttgaaa

SEQ ID NO: 39 - DNA, 17 bp, Arabidopsis thaliana
  source 1..17: mol_type = genomic DNA
tggaatggatcccaaac

SEQ ID NO: 40 - DNA, 17 bp, Arabidopsis thaliana
  source 1..17: mol_type = genomic DNA
ttgaatggatcccaaac

SEQ ID NO: 41 - DNA, 17 bp, Arabidopsis thaliana
  source 1..17: mol_type = genomic DNA
tggagtggatccccaac

SEQ ID NO: 42 - DNA, 17 bp, Arabidopsis thaliana
  source 1..17: mol_type = genomic DNA
tggagtggatccccaac

SEQ ID NO: 43 - DNA, 19 bp, synthetic construct
  source 1..19: mol_type = other DNA
ggagcaaagttctgggaag

SEQ ID NO: 44 - DNA, 20 bp, synthetic construct
  source 1..20: mol_type = other DNA
ctgaacatagctgtgaactg

SEQ ID NO: 45 - DNA, 19 bp, synthetic construct
  source 1..19: mol_type = other DNA
cctagctgtaagtaccatc

SEQ ID NO: 46 - DNA, 21 bp, synthetic construct
  source 1..21: mol_type = other DNA
gtttgttgagtttactgtctg

SEQ ID NO: 47 - DNA, 19 bp, synthetic construct
  source 1..19: mol_type = other DNA
gtttctgcaagatatgttg

SEQ ID NO: 48 - DNA, 20 bp, synthetic construct
  source 1..20: mol_type = other DNA
ttgtgtattcatgtagtgtg

SEQ ID NO: 49 - DNA, 20 bp, synthetic construct
  source 1..20: mol_type = other DNA
cgtctccacttcttcatggt

SEQ ID NO: 50 - DNA, 20 bp, synthetic construct
  source 1..20: mol_type = other DNA
ccttcacctgtgtaccaatg

SEQ ID NO: 51 - DNA, 19 bp, synthetic construct
  source 1..19: mol_type = other DNA
ctcgcaacaatacatctca

SEQ ID NO: 52 - DNA, 19 bp, synthetic construct
  source 1..19: mol_type = other DNA
gtctcagcagtaccgtgca

SEQ ID NO: 53 - DNA, 19 bp, synthetic construct
  source 1..19: mol_type = other DNA
gatctcagcagtaccgtaa

SEQ ID NO: 54 - DNA, 17 bp, synthetic construct
  source 1..17: mol_type = other DNA
atccacgccacggcagg

SEQ ID NO: 55 - DNA, 19 bp, synthetic construct
  source 1..19: mol_type = other DNA
agctgatcctcgtcatggt

(19) United States
(12) Patent Application Publication
(10) Pub. No.: US 2026/0035679 A1
(43) Pub. Date: Feb. 5, 2026

(54) GENOME EDITING TECHNIQUE

(71) Applicant: The University of Tokyo, Tokyo (JP)

(72) Inventors: Shin-ichi ARIMURA, Tokyo (JP); Ayako HOSODA, Tokyo (JP); Issei NAKAZATO, Tokyo (JP); Nobuhiro TSUTSUMI, Tokyo (JP); Hideki TAKANASHI, Tokyo (JP)

(73) Assignee: The University of Tokyo, Tokyo (JP)

(21) Appl. No.: 19/109,943

(22) PCT Filed: Aug. 31, 2023
(86) PCT No.: PCT/JP2023/031785

§ 371 Date: Mar. 7, 2025

(30) Foreign Application Priority Data
Sep. 9, 2022 (JP) 2022-143554

(51) Int. Cl. C12N 9/22; C12N 9/78; C12N 15/82

(52) U.S. Cl. CPC C12N 9/22; C12N 9/78; C12N 15/8205; C12Y 301/21004; C12Y 305/04005

(57) ABSTRACT

It is an object of the present invention to provide a method for modifying multiple DNAs encoding identical or similar proteins using TALE, when the multiple DNAs are present. More specifically, the present invention relates to a method for modifying multiple DNAs encoding identical or similar proteins, wherein the method comprises allowing the TALE portion of one type of TALE-modifier complex comprising at least one repeat sequence containing RVD (repeat variable di-residue) composed of amino acids that recognize or tolerate N, V, H, D, B, R, Y, M, W, S or K, to bind to the binding regions of the multiple DNAs.

A sequence listing in electronic ST.26 (XML file) format is filed with this application and incorporated herein by reference. The name of the ST.26 file is “P73148_SL.xml”; the file was created on Oct. 15, 2025; the size of the file is 56,382.

### TECHNICAL FIELD

The present invention relates to a genome editing technique using TALE (transcription activator-like effector).

### BACKGROUND ART

TALE has been identified as a transcription factor that is introduced into host cells, when the host plant is infected with the plant pathogenic bacterium Xanthomonas. When TALE is introduced into the host cells, it functions to control transcription in the cells, suppress immune responses, and induce an environment suitable for proliferation of Xanthomonas (Non Patent Literature 1 and Non Patent Literature 2). The DNA-binding domain of TALE has a structure in which 10 to 30 amino acid repeat sequences each consisting of approximately 34 amino acid residues are arranged in tandem, and binds to a target nucleotide sequence on the genome. The amino acid sequence that constitutes the repeat sequence consisting of approximately 34 amino acids has a variable region consisting of two amino acid residues called Repeat Variable Di-residue (RVD). The two amino acid residues that constitute the RVD determine which nucleotide in the target DNA sequence are recognized or tolerated (Non Patent Literature 3 and Non Patent Literature 4). The RVD corresponds to the 12th and 13th, or 13th and 14th amino acids from the N-terminus of the repeat sequence of the TALE protein.

Utilizing the specific DNA binding ability of TALE, several genome editing tools have been developed so far. For example, an artificial endonuclease in which an endonuclease is linked to the DNA-binding domain of TALE can be used as a sequence-specific endonuclease, TALEN (transcription activator-like effector nuclease), by designing the RVD to recognize or tolerate a desired nucleotide sequence (for example, Non Patent Literature 5). In addition, a fused body of TALE with cytidine deaminase (CD) or adenosine deaminase (ADA), which can modify double-stranded DNA, can be used to specifically modify a desired nucleotide [i.e., CD modifies C (cytosine) to U (uridine), and ADA modifies A (adenine) to I (inosine)] (Non Patent Literature 7, Non Patent Literature 12, and Patent Literature 1).

Genome editing tools using TALE have been improved in various ways even since then. When TALEN has been first developed, the DNA cleavage domain of FokI, which exhibits nuclease activity by dimerization, has been used as the nuclease domain of TALEN, and thus, a pair of TALENs that bind to each of the sense strand and the antisense strand needed to be prepared. Thereafter, compact TALEN has been developed, in which instead of the FokI nuclease domain, the catalytic region of bacteriophage-derived I-TevI is linked to TALE, so that the TALEN, as a monomer, can recognize and can cleave a target sequence (Non Patent Literature 8 and Patent Literature 2). Moreover, Sakuma et al. have modified the amino acid sequence of the DNA binding module of TALE, other than the RVD, and have developed TALEN (Platinum TALEN) that has higher activity than the conventional TALENs (Non Patent Literature 9). Furthermore, several reports have been published regarding the combination of amino acids in the RVD region in order to improve the binding stability of TALE with DNA (Non Patent Literature 10, Non Patent Literature 11, and Patent Literature 2).

In addition to TALE, the technique using CRISPR/Cas9 has also commonly used as a genome editing technique. CRISPR/Cas9 recognizes a sequence consisting of 20 nucleotides and edits the target sequence, but it may incorrectly edit a sequence similar to the target sequence, called an off-target. In contrast, TALE recognizes a sequence consisting of about 40 nucleotides, and so, off-target editing is less likely. However, on the other hand, in the case of using TALE, it is difficult to edit multiple similar sequences simultaneously.

### CITATION LIST

### Patent Literature

Patent Literature 1: WO2022/158561
Patent Literature 2: U.S. Pat. No. 20,130,117869 A1
Patent Literature 3: WO2011/072246

### Non Patent Literature

Non Patent Literature 1: Voytas and Joung, Science, 326:1491-1492, 2009
Non Patent Literature 2: Bogdanove et al., Current Opinion in Plant Biology, 13:394-401, 2010
Non Patent Literature 3: Boch et al., Science, 326:1509-1512, 2009
Non Patent Literature 4: Moscou and Bogdanove, Science, 326:1501, 2009
Non Patent Literature 5: Miller et al., Nature Biotechnology, 29:143-148, 2011
Non Patent Literature 6: Mok et al., Nature, 583:631-637, 2020
Non Patent Literature 7: Mok et al., Nature Communications, 13:4038 doi.org/10.1038/s41467-022-31745-y 2022
Non Patent Literature 8: Beurdeley et al., Nature Communications, 4:1762 DOI: 10.1038/ncomms2782, 2013
Non Patent Literature 9: Sakuma et al., Scientific Reports, 3:3379 DOI: 10.1038/strep03379, 2013
Non Patent Literature 10: Cong et al., Nature Communications, 3:968 DOI: 10.1038/ncomms1962, 2012
Non Patent Literature 11: Christian et al., PLOS One, 7: e45383, 2012
Non Patent Literature 12: Cho et al., Cell, 185:1764-1776, 2022

### SUMMARY OF INVENTION

### Technical Problem

In the genomes of many organisms, there is not only one gene that encodes a certain protein, but multiple genes, and in many cases, the nucleotide sequences thereof are similar sequences that are not completely identical to one another. For example, in multigenes or copy genes, it has often been found that the 3rd nucleotide of the codon for the same amino acid in the protein encoded by each gene is different for each multigene or copy gene. In addition, in plants, the genome is not only a 2n polyploid, but many are 3n or more polyploids, and further, each genome often encodes multiple target genes. Due to this functional redundancy, even if one specific gene sequence is genome-edited using the previous genome editing technique, the phenotype of the functional modification often does not clearly appear, which has been a problem.

In view of the aforementioned circumstances, it is an object of the present invention to provide a genome editing technique of simultaneously modifying a plurality of identical genes or similar genes using TALE (one type of TALE), when the plurality of identical genes or similar genes exist.

### Solution to Problem

The present inventors have attempted to achieve the aforementioned object by using TALE (transcription activator-like effectors) in which Repeat Variable Di-residue (RVD) has been modified to be a combination of specific amino acids.

In the case of homologous genes (homologs) or similar genes (homeologs) that exist in polyploid genomes, or a gene group existing as a gene family, their nucleotide sequences may not be completely identical to one another due to synonymous substitution and nonsynonymous substitution SNPs (Single Nucleotide Polymorphisms), even in the case of encoding proteins having similar functions. Therefore, when specific common regions of multiple genes in the gene group are simultaneously edited using TALE, there may be different nucleotides between individual genes in the gene group in the nucleotide sequence to which the TALE is to be bound. In I such cases, if a TALE having an RVD that can recognize or tolerate all nucleotides A, T, G or C, or multiple bases is constructed, it will become possible to carry out genome editing of simultaneously modifying multiple genes encoding proteins having similar functions, namely, multiple genes encoding proteins having homologous or similar amino acid sequences, in which the nucleotide sequences are slightly different from one another, by a single operation using a single genome editing enzyme.

It has been known that the β-tubulin gene TUB4 in the Arabidopsis thaliana genome causes a phenotype of superficial cell rows and twisted primary roots, when a nucleotide substitution causing Ser351Phe occurs (Ishida et al., Proceedings of the National Academy of Sciences, 104:8544-8549, 2007). Arabidopsis thaliana has 9 β-tubulin genes, and Ser351 is conserved in all of these 9 genes. The present inventors have introduced a nucleotide substitution from cytosine to thymine into the codon sequences encoding Ser351 in TUB1, TUB2, TUB3 and TUB4 out of the 9 genes, using nuclear-targeted TALE cytidine deaminase (nTALECD) (see WO2022/158561, etc.), so that they have attempted to induce a mutation of converting the 35 1st Ser to Phe or Leu. Among the TALE recognition sequences of TUB1, TUB2, TUB3 and TUB4 (sequences, to which the repeat sequence of the TALE left binds), the configuration of nucleotides at three positions were different among the genes. Thus, the present inventors have designed the TALE domain, so that the RVD that recognizes or tolerates these 3 nucleotides could become a combination of amino acids that recognizes N, namely, a combination of amino acids that recognize or tolerate A, T, G or C.

Using nTALECD having N-recognizing RVD, a nucleotide substitution from cytosine to thymine was introduced into the codon sequence encoding Ser351 of the Arabidopsis thaliana β-tubulin gene. As a result, in the T₁ generation, mutations were introduced into the 4 targeted β-tubulin genes (i.e., TUB1, TUB2, TUB3, and TUB4) in multiple individuals. Furthermore, among 5 non-targeted β-tubulin genes, individuals were found to also have mutations introduced into TUB5, TUB6, and TUB7. In contrast, in the case of using nTALECD, which was designed to introduce a nucleotide substitution specifically into TUB4, the mutations was introduced into the targeted TUB4 with high efficiency, while no mutation introduction was detected in other 8 β-tubulin genes, except one individual.

As described above, the present inventors have discovered for the first time that amino acids are arranged, so that the RVD, which is the RVD region of TALE and recognizes or tolerates nucleotides that are different among multiple gene sequences having identical functions, is allowed to correspond to N, and as a result, the editing of the multiple genes becomes possible, thereby completing the present invention. Based on the above-described findings, by appropriately using repeat sequences with RVDs that recognize or tolerate V (A, C or G), H (A, C or T), D (A, G or T), B (C, G or T), R (G or A), Y (C or T), M (A or C), W (A or T), S (C or G) or K (G or T), in addition to N, it becomes possible to expand the range of targets of gene editing.

Specifically, the present invention includes the following (1) to (14).
(1) A method for modifying multiple DNAs encoding identical or similar proteins, wherein
the method comprises allowing the TALE portion of one type of TALE-modifier complex comprising at least one repeat sequence containing RVD (repeat variable di-residue) composed of amino acids that recognize or tolerate N, V, H, D, B, R, Y, M, W, S or K, to bind to the binding regions of the multiple DNAs.
(2) A method for modifying multiple genes encoding identical or similar proteins in a cell, wherein
the method comprises introducing one type of TALE-modifier complex comprising at least one repeat sequence containing RVD composed of amino acids that recognize or tolerate N, V, H, D, B, R, Y, M, W, S or K, into the cell.
(3) A method for producing a cell, in which multiple genes encoding identical or similar proteins in the cell are modified, wherein
the method comprises introducing one type of TALE-modifier complex comprising at least one repeat sequence containing RVD composed of amino acids that recognize or tolerate N, V, H, D, B, R, Y, M, W, S or K, into the cell.
(4) The method according to any one of the above (1) to (3), wherein the nucleotide(s) recognized or tolerated by the RVD are nucleotide(s), in which when the nucleotide sequences of the multiple DNAs or genes are aligned, one or multiple nucleotides present at the same positions are different from the nucleotides of other DNAs or genes.
(5) The method according to any one of the above (1) to (3), wherein
the amino acids of the RVD are composed of RV, CS, VR, NA, S*, RH, RL or RT that recognize or tolerate N,
are composed of HC or KC that recognize M,
are composed of HS, HT, HV, KV or RC that recognize V, or
are composed of NT that recognize R or V, wherein
the “*” in S* indicates that the second digit of the RVD is a gap.
(6) The method according to any one of the above (1) to (3), wherein the modifier is whole or a part of an endonuclease, or whole or a part of a deaminase.
(7) The method according to the above (2) or (3), wherein the genes are nuclear genes, mitochondrial genes or plastid genes.
(8) The method according to the above (2), wherein the cell is a plant cell.
(9) The method according to the above (3), wherein the cell is a plant cell.
(10) A plant cell produced by the method according to the above (9).
(11) A seed or a plant, comprising the plant cell according to the above (10).
(12) A DNA-binding protein, comprising at least one repeat sequence of TALE, wherein
the RVD comprised in the repeat sequence is composed of RV, CS, VR, NA, S*, RH, RL or RT that recognize or tolerate N,
is composed of HC or KC that recognize M,
is composed of HS, HT, HV, KV or RC that recognize V, or
is composed of NT that recognize R or V, wherein
the “*” in S* indicates that the second digit of the RVD is a gap.
(13) The protein according to the above (12), wherein the RVD is RV that recognize or tolerate N.
(14) The protein according to the above (13), which is characterized in that it fuses with a functional protein.

It is to be noted that the preposition “to” used in the present description indicates a numerical value range including the numerical values located left and right of the preposition.

### Advantageous Effects of Invention

According to the present invention, it becomes possible to simultaneously perform identical modifications on the gene sequences of multiple genes having identical functions, even in a case where the multiple gene sequences are not completely identical to one another.

### BRIEF DESCRIPTION OF DRAWINGS

FIGS. 1a and 1b show the design outline of TALE+ that simultaneously targets duplicated genes. FIG. 1a shows the alignment of some DNA nucleotide sequences of 9 β-tubulin genes (TUB1 to TUB9) present in the Arabidopsis thaliana genome. In the DNA-recognizing domains of the left and right of a TALE pair that specifically targets TUB4 (TUB4-specific TALE pair), which is composed of a generally used repeat sequence with RVD, 8 repeats (corresponding to the highlighted nucleotides) out of a total of 35 repeats were substituted with recognition repeats (N) (TALE+ pair 8N (3+5): targets 4 gene loci, TUB1, TUB 2, TUB 3, and TUB 4). The underlined nucleotides are those that do not correspond to the repeats of the TALE+ pair 8N (3+5) and are different among individual TUBs. The nucleotide sequences shown in FIG. 1a are, from top to bottom, SEQ ID No: 30, SEQ ID No: 31, SEQ ID No: 32, SEQ ID No: 33, SEQ ID No: 34, SEQ ID No: 35, SEQ ID No: 36, SEQ ID No: 37, and SEQ ID No: 38. FIG. 1b shows examples (TALE left sequences of 8N) of the design of DNA-binding sequences of TALECD using an N-recognizing RVD (RV). The nucleotides that are identical in all of the 4 target genes are recognized using a general RVD repeat that specifically recognizes 4 nucleotides, and the sites that are different in terms of the configuration of nucleotides in the 4 genes are recognized or tolerated using the N-recognizing RVD (RV). The nucleotide sequences shown in FIG. 1b are, from top to bottom, SEQ ID No: 39, SEQ ID No: 40, and SEQ ID No: 41, SEQ ID No: 42.

FIG. 2 shows the waveform of the Sanger sequence of a 15-base-long target window flanked between the left and right TALE recognition sequences in the target locus of a T₁ individual (#17) in which mutation introduction was confirmed simultaneously in TUB1, TUB2, TUB3 and TUB4. The target cytosine nucleotide (highlighted, blue waveform) is partially or completely substituted with thymine (green waveform).

FIG. 3 shows the genotypes of the first generation (T₁) transformed plants on 14 days after a low temperature treatment on seeds. h/c: Hetero or chimera of a wild-type nucleotide (cytosine) and a substituted nucleotide (thymine); homo: Complete substitution.

FIG. 4 shows the percentage of T₁ individuals by the number of β-tubulin genes in which mutations were simultaneously introduced into the target nucleotides (white: TUB4-specific, n=8; black: TALE+ 8N, n=22).

FIGS. 5a and 5b show a schematic view of a step of constructing an nTALECD expression vector. FIG. 5a shows an outline of an assembly of the DNA-binding domains of TALECD. Module plasmids comprising each repeat sequence are combined with one another to construct an intermediate vector having 1 to 4 units of consecutive repeat sequences. Next, the intermediate vectors are connected with one another to construct an entry vector having the coding sequence of a fused protein of a full-length repeat sequence, cytidine deaminase (CD half) and uracil glycosylase inhibitor (UGI). FIG. 5b shows the cloning method of the tandem expression construct of TALECD. A binary vector is constructed by a multi-site LR reaction using the two types of entry vectors expressing the full-length left and right TALECDs constructed in FIG. 5a, an entry vector having a nuclear localization signal sequence (NLS), a promoter sequence, a terminator sequence, etc., and a destination vector.

### DESCRIPTION OF EMBODIMENTS

Hereafter, the embodiments for carrying out the present invention will be described.

A first embodiment relates to a method for modifying multiple DNAs encoding identical or similar proteins, wherein the method comprises allowing the TALE portion of one type of TALE-modifier complex comprising at least one repeat sequence containing RVD (repeat variable di-residue) composed of amino acids that recognize or tolerate N, V, H, D, B, R, Y, M, W, S or K, to bind to the binding regions of the multiple DNAs (DNA regions to which the TALE portion binds) (hereinafter also referred to as “the DNA modification method according to the present embodiment”). The “TALE-modifier complex” refers to a fused body (or a bound body or a linked body) of a TALE and a modifier. Herein, “modification” of DNA includes not only changing the structure of a DNA sequence, but also controlling the transcriptional activity of DNA that encodes a protein, such as, for example, activating or suppressing the functions of a promoter, an enhancer, a silencer, etc., and epigenetic control. Moreover, examples of “changing the structure of a DNA sequence” may include, but are not particularly limited to, introducing a substitution (change), insertion, deletion or addition of one or multiple nucleotides into a DNA sequence, a double strand cleavage of a DNA strand, introducing a double strand cleavage and binding, etc. into a DNA sequence to change the structure of the DNA sequence, and also, modifying the nucleotides that constitute DNA to change the structure of the sequence, for example, methylating one or multiple nucleotides in a DNA sequence or introducing nicks into a DNA strand, etc. so as to modify the nucleotides that constitute a DNA strand or DNA.

In the TALE-modifier complex of the present embodiment, the “modifier” refers to a factor having the function or activity of “modifying” the aforementioned DNA. Examples of the factor that induces modification of the sequence structure of DNA may include, but are not particularly limited to, enzymes that change the sequence structure of DNA, such as endonuclease and deaminase. Examples of the factor that modifies nucleotides may include, but are not particularly limited to, enzymes such as DNA methylase, DNA glycosylase, and nickase. Examples of the endonuclease may include a FokI nuclease domain, and I-TevI derived from bacteriophage. Examples of the deaminase may include the cytidine deaminase domain of DddA of Burkholderia cenocepacia (hereinafter also referred to as DddAₜₒₓ), which modifies cytosine (C) in DNA to be uridine (U) (see WO2022/158561 for details), and adenosine deaminase, which modifies adenine (A) to inosine (I) (see Cho et al., Cell, 185:1764-1776, 2022). Furthermore, examples of the factor that controls the transcriptional activity of DNA encoding a protein may include a transcriptional activator and a transcriptional repressor and some domains thereof, as well as epigenetic regulators such as DNA methylase and histone modification enzymes (e.g., histone acetyltransferase, histone deacetylase, histone methyltransferase, etc.) and some domains thereof.

The DNA modification method according to the present embodiment can be used not only for modification of genes present in cells, but also for DNA modification in a cell-free system. In the present embodiment, “DNA” includes, for example, genomic DNA as well as cDNA, and when DNA modification is performed in a cell-free system, for example, DNAs contained in a genomic DNA library or a cDNA library, etc. may be used as “multiple DNAs.” The “target sequence” is a DNA region to which a TALE portion binds. When DNA modification is performed in a cell-free system, a TALE-modifier complex is mixed with an aggregation of multiple DNAs (for example, a genomic DNA library, a cDNA library, etc.), so that the TALE portion of the TALE-modifier complex is allowed to come into contact with the DNA. When the TALE portion of the TALE-modifier complex binds to the target region of DNA, the target base present in the vicinity thereof is modified by the modifier.

In the present embodiment, the “protein” includes not only a full-length protein, but also a portion of such a full-length protein that has a specific function, such as a protein domain (a portion of the sequence or structure of a protein that has a function: for example, an EF-hand protein domain, a zinc finger domain, etc.). In addition, the “identical proteins” are “proteins” that have the same function and activity as each other, and have a 100% identical amino acid sequence to each other, whereas the “similar proteins” are “proteins” that have the same function and activity as each other, and have a 90% or more, 95% or more, or 99% or more identical amino acid sequence to each other.

Moreover, the “multiple DNAs” mean DNAs in which all of the multiple DNAs encode “identical proteins” or “similar proteins.” In this case, when the “DNAs” that encode the identical or the similar proteins are genes (genomic DNAs), the “multiple DNAs” mean multiple genes (genomic DNAs) whose mRNA sequences transcribed from the multiple genes (genomic DNAs) are not identical to one another. The present multiple genes (genomic DNAs) are not particularly limited, and examples thereof may include genes that constitute a gene family, duplicated genes, and copy genes.

The nucleotide(s), which the RVD of the present embodiment (i.e., the RVD comprised in at least one repeat sequence of the TALE) recognizes or tolerates, are nucleotide(s), in which when the nucleotide sequences of the multiple DNAs are aligned, one or multiple nucleotides present at the same position are different from the nucleotides of other DNAs (nucleotides present at the same position).

When the DNA modification method according to the present embodiment is applied to a gene (genomic DNA) in a cell, a first embodiment is a method for modifying a gene (genomic DNA) that encodes an identical or similar protein in a cell, wherein the method comprises introducing into the cell one type of TALE-modifier complex comprising at least one repeat sequence containing RVD composed of amino acids that recognize or tolerate N, V, H, D, B, R, Y, M, W, S or K (hereinafter also referred to as “the gene modification method according to the present embodiment”).

As mentioned above, living organisms have multiple genes that encode identical proteins, and the sequences of the multiple genes may not be completely identical to one another. Therefore, when such multiple genes are modified using TALE, the recognition sequence to which the TALE binds may be different among gene copies. The present embodiment will be explained using the following case.

It is assumed that Gene A has six nucleotide sequences of Genes A-1 to Gene A-6. The binding regions of the TALE for modifying Gene A are aligned from Gene A-1 to Gene A-6, as shown below.

Gene A-1
(SEQ ID No: 1)
GGA TCT TAT CAT GGT
Gene A-2
(SEQ ID No: 2)
GGA TCC TAT CAT GGT
Gene A-3
(SEQ ID No: 3)
GGA TCA TAT CAT GGT
Gene A-4
(SEQ ID No: 4)
GGA TCG TAT CAT GGT
Gene A-5
(SEQ ID No: 5)
GGA TCC TAT CAT GGT
Gene A-6
(SEQ ID No: 6)
GGA TCT TAT CAT GGT

Among the above six gene sequences, the underlined nucleotides are different from one another. However, the amino acid sequences encoded by these sequences are all identical to one another, and it is GSYHG (SEQ ID No: 7). In such a case, when an identical modification is introduced into Gene A or a Gene A product (protein) in a cell, using TALE, according to the conventional method, it has been necessary to prepare four TALEs, in which RVDs of repeat sequences recognizing the underlined nucleotides are each constituted with, for example, NG (Asn-Gly) that recognizes T, HD (His-Asp) that recognizes C, NI (Asn-I1e) that recognizes A, or NN (Asn-Asn) that recognizes G. In contrast, as in the method of the present embodiment, if the RVD that recognizes the underlined nucleotide is made to have an amino acid configuration that recognizes or tolerates N (wherein “N” represents A, T, G, or C), it is possible to produce a TALE that binds to all recognition sequences of Genes A-1 to A-6. Furthermore, if the RVD that recognizes the underlined nucleotide is made to have an amino acid configuration that recognizes R (wherein “R” represents A or G), a TALE that binds to the recognition sequences of Gene A-3 and Gene A-4 can be produced, and if it is made to have an amino acid configuration that recognizes Y (wherein “Y” represents T or C), a TALE that binds to the target sequences of Gene A-1, Gene A-2, Gene A-5 and Gene A-6 can be produced. Thus, in the above-described case, the RVD of the present embodiment is characterized in that, when Gene A-1, Gene A-2, Gene A-3, Gene A-4, Gene A-5 and Gene A-6 are aligned, the 6th nucleotide of SEQ ID Nos: 1 to 6, namely, the present RVD recognizes nucleotides that are different among Gene A-1, Gene A-2, Gene A-3, Gene A-4, Gene A-5 and Gene A-6.

The bond between the TALE portion and the modifier in the TALE-modifier fused body of the present embodiment may be either a direct bond by a peptide bond or the like, or an indirect bond via a linker or the like. In addition, the TALE portion of the TALE-modifier fused body, namely, the DNA-binding domain, comprises a repeat structure of an amino acid sequence consisting of about 34 amino acids (hereinafter, the amino acid sequence consisting of about 34 amino acids will also be referred to as a “repeat sequence”). The TALE portion used in the present embodiment may comprise generally 2 or more, preferably 6 or more, more preferably 16 or more, and also, may comprise generally 36 or less, preferably 24 or less, more preferably 20 or less repeat sequences.

Herein, the “repeat sequence” may be, for example, the amino acid sequence as set forth in the following SEQ ID No: 8, SEQ ID No: 9, SEQ ID No: 10, SEQ ID No: 11, SEQ ID No: 12 or SEQ ID No: 13, or may also be an amino acid sequence, in which a deletion, substitution or addition has occurred in the amino acid sequence as set forth in SEQ ID No: 8, SEQ ID No: 9, SEQ ID No: 10, SEQ ID No: 11, SEQ ID No: 12 or SEQ ID No: 13, and which has a sequence identity of 80% or more, preferably 90% or more, to the amino acid sequence as set forth in SEQ ID No: 8, SEQ ID No: 9, SEQ ID No: 10, SEQ ID No: 11, SEQ ID No: 12 or SEQ ID No: 13, respectively.

### Examples of the Repeat Sequence

LTPDQVVAIASXXGGKQALETVQRLLPVLCQDHG (SEQ ID No: 8; “XX” represent 2 amino acids that constitute the RVD.)
LTP(D/E/A) QVVAIASXXGGKQALETVQRLLPVLCQ (D/A) HG (SEQ ID No: 9: “XX” represent 2 amino acids that constitute the RVD, “D/E/A” represents D, E or A, and “D/A” represents D or A.)
LTPDQVVAIASXXGGKQAL (E/A) T (V/M) Q (R/A) LLPVLCQDHG (SEQ ID No: 10: “XX” represent 2 amino acids that constitute the RVD, “E/A” represents E or A, “V/M” represents V or M, and “R/A” represents R or A.)
LTPEQVVAIASXXGGRPALE (SEQ ID No: 11: “XX” represent 2 amino acids that constitute the RVD.)
LTPDQVVAIASXXGGKQALES (SEQ ID No: 12: “XX” represent 2 amino acids that constitute the RVD.)
LTPNQVVAIASXXGGKQALE (SEQ ID No: 13: “XX” represent 2 amino acids that constitute the RVD.)

It is to be noted that the repeat sequence shown in any of SEQ ID No: 9, SEQ ID No: 10 or SEQ ID No: 11 is sometimes used as a single repeat at the C-terminus of the DNA-binding domain of a TALE, and that this repeat sequence is a shorter sequence (approximately 20 amino acids) than other repeat sequences (see, for example, WO2011/072246, etc.).

There are several reports regarding the correspondence between the amino acids that constitute the RVD and the recognized nucleotide (for example, Patent Literature 2, Non Patent Literature 3, Non Patent Literature 4, etc.). For example, it has been reported that HD recognizes C, NG recognizes T, NI recognizes A, NN recognizes G or A, NS recognizes A, T, C or G, HG recognizes T, IG recognizes T, HA recognizes C, ND recognizes C, NK recognizes G, HI recognizes C, HN recognizes G, NA recognizes G, SN recognizes G or A, and YG recognizes T.

Moreover, the RVD may be composed of RV, CS, VR, NA, S* (“*” indicates that the second digit of the RVD is a gap), RH, RL or RT, which recognize or tolerate N: may be composed of HC or KC, which recognize M: may be composed of HS, HT, HV, KV or RC, which recognize V; or may be composed of NT, which recognize R or V.

The gene modification method according to the present embodiment and a method for producing cells (as described later) can be applied to the genes or cells of both prokaryotes and eukaryotes.

Regarding genes, in eukaryotes, the method can be applied not only to nuclear genes but also to mitochondrial genes and plant plastid (e.g., chloroplast) genes. In order to specifically modify the target nucleotide of DNA in a nuclear gene, a mitochondrial gene or a plastid gene, it is necessary to allow the modifier to recognize the target nucleotide. For this purpose, a TALE-modifier fused body is introduced into the nucleus, mitochondria or plastid. More specifically, for example, DNA encoding a TALE-modifier fused body protein may be introduced into nuclear genomic DNA (i.e., incorporated into nuclear genomic DNA), and the TALE-modifier fused body protein expressed in the cytoplasm may be transported (introduced) into the nucleus, plastid or mitochondria. In this case, it is desirable that DNA encoding a fused body, in which various types of signal peptides (a nuclear transport signal peptide, a mitochondrial transport a signal peptide, or a plastid transport signal peptide) are added (bound) to the TALE-modifier fused body protein, is introduced into the nuclear genomic DNA.

An example of a method for transporting a TALE-modifier fused body protein into the nucleus may be a method of fusing a nuclear transport (localization) signal (nuclear localization signal/sequence: NLS) peptide with a TALE-modifier fused protein and then allowing the fused body to express. Examples of the nuclear transport signal peptides that can be used in the present embodiment may include, but are not limited to, the SV40 large T antigen NLS peptide (PKKKRKV, SEQ ID No: 14), the nucleoplasmin NLS peptide (AVKRPAATKKAGQAKKKKLD, SEQ ID No: 15), the EGL-13 NLS peptide (MSRRRKANPTKLSENAKKLAKEVEN, SEQ ID No: 16), the c-Myc NLS peptide (PAAKRVKLD, SEQ ID No: 17), and the TUS protein NLS peptide (KLKIKRPVK, SEQ ID No: 18). Other than these nuclear transport signal peptides, there are other available nuclear transport signal peptides, and for example, please refer to the NLSdb (https://rostlab.org/services/nlsdb/browse/signals), a database of nuclear transport signals.

An example of a method for transporting a TALE-modifier fused body protein into the mitochondria may be a method of fusing a mitochondrial transport signal peptide (e.g., a peptide that does not have a clear higher-order structure or sequence homology, but is characterized in that, for example, a basic amino acid and multiple hydrophobic amino acids appear alternately, etc.) with a TALE-modifier fused protein and then allowing the fused body to express. Examples of the mitochondrial transport signal peptides that can be used in the present embodiment may include: in the case of animal cells, human ATPase Fb1 subunit-derived signal peptide (Payam et al., EMBO Mol Med, 6:458-466, 2014) and human cytochrome c oxidase subunit 8 (Bacman et al., Gene Therapy, 17:713-720, 2010): and in the case of plant cells, for example, Arabidopsis thaliana ATPase δ′ subunit-derived signal peptide (MFKQASRLLS RSVAAASSKS VTTRAFSTEL PSTLDS, SEQ ID No: 19), rice ALDH2a gene product-derived signal peptide (MAARRAASSL LSRGLIARPS AASSTGDSAI LGAGSARGFL PGSLHRFSAA PAAAATAAAT EEPIQPPVDV KYTKLLINGN FVDAASGKTF ATVDP, SEQ ID No: 20), and pea cytochrome c oxidase Vb-3-derived signal peptide (MWRRLFTSPH LKTLSSSSLS RPRSAVAGIR CVDLSRHVAT QSAASVKKRV EDVV, SEQ ID No: 21), as well as Arabidopsis thaliana ATPase β subunit-derived signal peptide and chaperonin CPN-60-derived signal peptide (Logan et al., Journal of Experimental Botany, 50 865-871 2000), and a rice F1F0-ATPase inhibitor protein signal peptide (Nakazono et al., Plant, 210 188-194, 2000).

An example of a method for transporting a TALE-modifier fused body protein into the plastid may be a method of fusing a plastid transport signal peptide (e.g., a peptide that does not have a clear higher-order structure or sequence homology, but is, for example, rich in basic amino acids and multiple hydrophobic amino acids and has few acidic amino acids, and exhibits a function of being selectively transported specifically to chloroplasts or plastids by being added to the N-terminus of the amino acid sequence of a protein, etc.) with a TALE-modifier fused protein and then allowing the fused body to express. The plastid transport signal peptide that can be used in the present embodiment is preferably, for example, a signal peptide possessed by a protein localized in a plant plastid. Examples of the preferred signal peptide may include, but are not limited to, protein-derived signal peptides such as RECA1, RBCS, CAB, NEP, SIG1 to 5, and GUN2 to 5, as well as nuclear-encoded chloroplast ribosomal protein-derived signal peptides such as RPL12 and RPS9, nuclear-encoded chloroplast tRNA aminoacyl transfer factor-derived signal peptides, nuclear-encoded chloroplast heat shock protein-derived signal peptides, other protein-derived signal peptides such as FtsZ, FtsH, MinC, MinD, and MinE, nuclear-encoded chloroplast photosynthesis-related enzyme complex enzyme group-derived signal peptides, nuclear-encoded plastid lipid metabolic enzyme group-derived signal peptides, and nuclear-encoded thylakoid component protein group-derived signal peptides. For details of plastid transport signal peptides, see, for example, von HEIJNE et al., European Journal of Biochemistry, 180, 535-545, 1989, etc.

In some cases, there can also be used a method of directly introducing plasmid DNA encoding a TALE-modifier fused body protein, mRNA, and the TALE-modifier fused body protein itself, into a cell (the introduction method may include, for example, a viral method, a particle gun method, a PEG method, a cell membrane permeable peptide method, etc.).

The DNA encoding the TALE-modifier fused body protein (which may also include a protein to which a signal peptide bind) according to the present embodiment can be produced by a method known in the present technical field. Alternatively, the DNA encoding the present TALE-modifier fused body protein may also be produced using a commercially available kit. More specifically, with regard to production of the TALE portion, for example, a kit based on a Golden Gate method (Cermak et al., Nucleic Acids Res. 39: e82, 2011) or a kit based on a modified method thereof (Sakuma et al., Genes Cells 18:315-326, 2013), such as a FusX TALEN assembly system (Addgene kit #1000000063), etc. may be used. These kits can be obtained, for example, from Addgene, etc.

A second embodiment relates to a method for producing a cell, in which multiple genes encoding identical or similar proteins in the cell are modified, wherein the method comprises introducing one type of TALE-modifier fused body comprising at least one repeat sequence containing RVD composed of amino acids that recognize or tolerate N, V, H, D, B, R, Y, M, W, S or K, into the cell (hereinafter also referred to as “the cell production method according to the present embodiment). The cells produced in the second embodiment may be used to produce mutant individuals or mutant strains of the organism from which the cells are derived. Therefore, the present embodiment includes cells produced by the method according to the second embodiment, as well as individual organisms comprising the cells. For example, taking a plant as an example, the present embodiment includes plant cells produced by the method according to the second embodiment, and seeds or plants (plant adult bodies) comprising the plant cells.

The “cells” in the present embodiments (the first and the second embodiments) may be either cells of prokaryotes or cells of eukaryotes. The cells of prokaryotes are not particularly limited, and for example, there may be used Escherichia bacteria (Escherichia coli, etc.), Bacillus bacteria (Bacillus subtilis, etc.), and Agrobacterium (for example, Rhizobium bacteria (e.g., Rhizobium tumefacience and Rhizobium rhizogenes), etc.). The cells of eukaryotes are not particularly limited, and may be, for example, yeasts [Saccharomyces cerevisiae, etc.], established mammalian cell lines, primary cultured cells collected from living bodies of mammals (mouse embryonic fibroblasts MEF, primary cultured neural cells, etc.), ES cells, iPS cells, and further, plant cells. The plant cells may be cultured cells derived from plants, as well as cells derived from plants (e.g., cells derived from ovules), and the plant cells may further include plant cells derived from plants with various forms, such as suspension culture cells, protoplasts, leaf segments, callus, immature embryos, pollen, etc.

The mammals are not particularly limited, and examples thereof may include mice, rats, hamsters, guinea pigs, rabbits, swines, bovines, goats, horses, sheep, dogs, cats, and humans or non-human primates (e.g., monkeys, cynomolgus monkeys, rhesus monkeys, marmosets, orangutans, chimpanzees, etc.). Examples of organisms other than the mammals may include nematodes (C. elegans), fish (zebrafish), and amphibians (Xenopus laevis and Xenopus tropicalis).

The plants are not particularly limited, and in the case of seed plants, any seed plants may be used. If daring to give some examples, examples of the plants that can be used herein may include: gramineous plants, such as rice, wheat, corn, barley, rye, and sorghum; and cruciferous plants, for example, plants belonging to genus Alyssum, genus Arabidopsis (Arabidopsis thaliana, etc.), genus Armoracia (horseradish, etc.), genus Aurinia, genus Brassica [Chinese flat cabbage, mustard green, Brassica juncea, rapeseed, Brassica rapa ssp., hagoromokanran (kale), flowering kale, cauliflower, cabbage, brussels sprouts (komochikaran), broccoli, bok choy, turnip greens mustard leaves, oilseed rape, Chinese cabbage, Japanese mustard spinach, turnip, etc.], genus Camelina, genus Capsella, genus Cardamine, genus Coronopus, genus Diplotaxis, genus Draba, genus Eruca (Rucola, etc.), genus Hesperis, genus Hirschfeldia, genus Iberis, genus Ionopsidium, genus Lepidium, genus Lobularia, genus Lunaria, genus Malcolmia, genus Matthiola, genus Nasturtium, genus Orychophragmus, genus Raphanus (Japanese radish, Raphanus sativus var. sativus, etc.), genus Rapistrum, genus Rorippa, genus Sisymbrium, genus Thlaspi, and genus Eutrema (Japanese wasabi mustard, etc.). Furthermore, other examples of the plants that can be used herein may include: solanaceous plants, such as tomato, potato, pepper, shishito pepper, and petunias: Asteraceae plants, such as sunflower and dandelion: Convolvulaceae plants, such as bindweed and sweet potato: araceous plants, such as konjak, taro, Colocasia esculenta, and Colocasia esculenta: leguminous plants, such as soybeans, adzuki beans, and green beans: cucurbitaceous plants, such as pumpkin, cucumber, and melon: and amaryllidaceous plants, such as onion, green onion, and garlic. The plant-derived cells include not only cultured cells derived from plants, but also cells in plant bodies. The plans further include plant cells derived from plants with various forms, such as suspension culture cells, protoplasts, leaf segments, callus, immature embryos, pollen, etc.

A third embodiment relates to a DNA-binding protein, comprising at least one repeat sequence of TALE, wherein
the RVD comprised in the repeat sequence is composed of RV, CS, VR, NA, S*, RH, RL or RT that recognize or tolerate N,
is composed of HC or KC that recognize M,
is composed of HS, HT, HV, KV or RC that recognize V, or
is composed of NT that recognize R or V.

The protein according to the third embodiment is characterized in that it comprises at least one novel RVD that recognizes or tolerates multiple types of nucleotides. The TALE portion of the protein of the present embodiment, namely, the DNA-binding domain, comprises a repeating structure of an amino acid sequence consisting of about 34 amino acids (hereinafter, an amino acid sequence consisting of about 34 amino acids is also referred to as a “repeat sequence”). The TALE portion used in the present embodiment may comprise generally 2 or more, preferably 6 or more, more preferably 16 or more repeat sequences, and also, may comprise generally 36 or less, preferably 24 or less, more preferably 20 or less repeat sequences.

Herein, the “repeat sequence” may be, for example, the amino acid sequence as set forth in the following SEQ ID No: 8, SEQ ID No: 9, SEQ ID No: 10, SEQ ID No: 11, SEQ ID No: 12 or SEQ ID No: 13, or may also be an amino acid sequence, in which a deletion, substitution or addition has occurred in the amino acid sequence as set forth in SEQ ID No: 8, SEQ ID No: 9, SEQ ID No: 10, SEQ ID No: 11, SEQ ID No: 12 or SEQ ID No: 13, and which has a sequence identity of 80% or more, preferably 90% or more, to the amino acid sequence as set forth in SEQ ID No: 8, SEQ ID No: 9, SEQ ID No: 10, SEQ ID No: 11, SEQ ID No: 12 or SEQ ID No: 13, respectively.

The protein according to the third embodiment may be fused (or bound) with another functional protein, namely, a protein (or polypeptide) having a function or activity different from the TALE. The bond between the protein according to the third embodiment and the functional protein may be either a direct bond by a peptide bond or the like, or an indirect bond via a linker or the like. The functional protein may be whole or a part of a protein having the function of modifying a nucleic acid sequence. Other examples of the functional protein may include whole or a part of a transcriptional activity regulator (a transcriptional activator or a transcriptional repressor, etc.), whole or a part of an epigenetic regulator, and whole or a part of a fluorescent protein, a luminescent protein and a pigment protein. Herein, the term “a part” means, for example, a part that exhibits a function of interest by itself, or exhibits a function of interest by forming a dimer.

A fourth embodiment relates to a nucleic acid (DNA, etc.) encoding the TALE-modifier fused body used in the first embodiment and the second embodiment, or the protein according to the third embodiment or a fused body of the present protein and a functional protein (hereinafter, these fused bodies or proteins are also referred to as “the proteins according to the present embodiment”). The TALE-modifier fused body used in the first embodiment and the second embodiment, and the protein according to the third embodiment and a fused body of the present protein and a functional protein, can be prepared by incorporating each of the nucleic acids encoding these fused bodies (the nucleic acids according to the fourth embodiment) into a suitable expression vector, then transforming or transfecting suitable host cells with the expression vector, then culturing the resulting cells in a suitable medium, and then allowing these proteins to express therein, followed by purifying them.

As host cells allowed to express the proteins according to the present embodiment, for example, bacterial cells (e.g., Escherichia coli B strain, E. coli K12 strain, Corynebacterium ammoniagenes, C. glutamicum, Serratia liquefaciens, Streptomyces lividans, Pseudomonas putida, etc.), molds (e.g., Penicillium camembertii, Acremonium chrysogenum, etc.), animal cells, plant cells, baculovirus/insect cells, or yeast cells (e.g., Saccharomyces cerevisiae and Pichia pastoris, etc.) are used, and the proteins according to the present embodiment can be expressed in these cells.

As expression vectors for expressing the proteins according to the present embodiment, vectors suitable for various types of host cells can be used. The present expression vectors can also be used in a case where proteins are expressed in cells by the methods according to the first embodiment and the second embodiment. Examples of the expression vector that can be used herein may include: pBR322, pBR325, pUC118, pET, etc. (Escherichia coli hosts): pEGF-C, pEGF-N, etc. (animal cell hosts): pVL1392, pVL1393, etc. (insect cell hosts, baculovirus vectors): and pG-1, Yep13, pPICZ, etc. (yeast cell hosts); and binary vectors for plant cells (pBG, pBI, pGreen, pCAMBIA, PLC, pSB11, pSB200, and pRI). These expression vectors each have a replication origin, a selective marker, and a promoter, which are suitable for each vector. These expression vectors may also have an enhancer, a transcription termination sequence (terminator), a ribosome binding site, a polyadenylation signal, etc., as necessary. Further, in order to facilitate purification of the expressed polypeptide, a nucleotide sequence for fusing a FLAG tag, a His tag, an HA tag, a GST tag, etc. with the polypeptide to express it may be inserted into such an expression vector.

Such an expression vector can be produced by a method known to a person skilled in the art, using a commercially available kit, as appropriate. In addition, the expression vector according to the present embodiment is preferably isolated or purified.

When the expressed protein is extracted from cultured cell masses or cultured cells, the cell masses or the cultured cells are collected by a known method after completion of the culture, and the collected cell masses or cells are then suspended in a suitable buffer. Thereafter, the suspension is subjected to ultrasonic wave, lysozyme and/or freezing-thawing, etc., so that the cell masses or cells are disintegrated. Thereafter, the resultant is subjected to centrifugation or filtration to obtain a soluble extract. In particular, when cultured cells are used as hosts, it is desirable to obtain a protein expressed in a culture supernatant by recovering the supernatant. An appropriate combination of known separation and/or purification methods is applied to the obtained extract or the culture supernatant, so as to obtain a protein of interest. Examples of the known separation and/or purification methods that can be used herein may include: methods of utilizing solubility, such as salting-out or a solvent precipitation method: methods of mainly utilizing a difference in molecular weights, such as a dialysis method, an ultrafiltration method, a gel filtration method, or SDS-PAGE: methods of utilizing a difference in electric charges, such as ion exchange chromatography: methods of utilizing specific affinity, such as affinity chromatography (for example, methods, in which when a polypeptide is expressed together with a GST tag, a glutathione-bound carrier resin is used, when a polypeptide is expressed together with a His tag, a Ni-NTA resin or a Co-based resin is used, when a polypeptide is expressed together with a HA tag, an anti-HA antibody resin is used, and when a polypeptide is expressed together with a FLAG tag, an anti-FLAG antibody-bound resin or the like is used); methods of utilizing a difference in hydrophobicity, such as reverse phase high performance liquid chromatography: and methods of utilizing a difference in isoelectric points, such as an isoelectric focusing method.

The disclosures of all publications cited in the present description are incorporated herein by reference in their entirety. In addition, throughout the present description, when singular terms such as “a,” “an,” and “the” are used, these terms include not only single items but also multiple items, unless otherwise clearly specified from the context that it is not the case.

Hereinafter, the present invention will be further described in the following examples. However, these examples are only illustrative examples of the embodiments of the present invention, and thus, are not intended to limit the scope of the present invention.

### EXAMPLES

### 1. Materials and Methods

### 1-1. Production of N-Recognizing Module Plasmids

Using, as templates, module plasmids (p1HD/#50664, p2HD/#50668, p3HD/#50672, and p4HD/#50676) included in the Platinum Gate TALEN kit (Addgene, Kit #1000000043), PCR was performed with the primer sets listed in Table 1 to produce amplicons in which the HD code was changed to the RV code. The produced amplicons were then subjected to In-Fusion HD Cloning Reaction (Takara), together with the original vectors cleaved with Pvu I, to produce the RV module plasmids of p1-4. In addition, similar PCR was performed using, as a template, an entry vector (E1_pENTR_L1-L4_NI_G1397-DddtoxA-N/#171727) having the RVD repeat sequence of the C-terminus of the DNA-binding domain. The same vector was treated with the restriction enzymes Kpn I and Xba I, and the purified linearization vector and the purified PCR product were used to perform In-Fusion HD Cloning Reaction. With regard to the resulting plRV, p2RV, p3RV and p4RV module plasmids and the entry vector (E1_pENTR_L1-L4_RV_G1397-DddtoxA-N) having the RV repeat sequence of the C-terminus, the DNA sequences of the RVD repeat portions were confirmed by Sanger sequencing (outsourced to Eurofins Genomics).

TABLE 1

| Primer Name | Primer Sequence (5' to 3') | Purpose |
|---|---|---|
| RV_Fw1 | TCGCTATTACGCCAGCTG (SEQ ID No: 22) | Construction of |
| RV_Rv1 | CACCCTTGACGCGATTGCAACCACC | p1~4RV vector |
| | (SEQ ID No: 23) | |
| RV Fw2 | AATCGCGTCAAGGGGGGGGGAAAGC | Construction of |
| | (SEQ ID No: 24) | |
| RV_Rv2 | GATTCATTAATGCAGCTGGC (SEQ ID No: 25) | p1~4RV vector |
| lastRV_Fw1 | TATAGGGCGAATTGGGTACCG (SEQ ID No: 26) | Construction of |
| lastRV_Rv1 | TCTGCCCCCCACCCTGGATGCAATAGCCACTAC | lastRV vector |
| | (SEQ ID No: 27) | |
| lastRV_Fw2 | AGGGTGGGGGGCAGACCCGCAC | Construction of |
| | (SEQ ID No: 28) | |
| lastRV Rv2 | TGTGGAGCTGAGATCTG (SEQ ID No: 29) | lastRV vector |

### 1-2. Design of DNA-binding Domain of TALECD

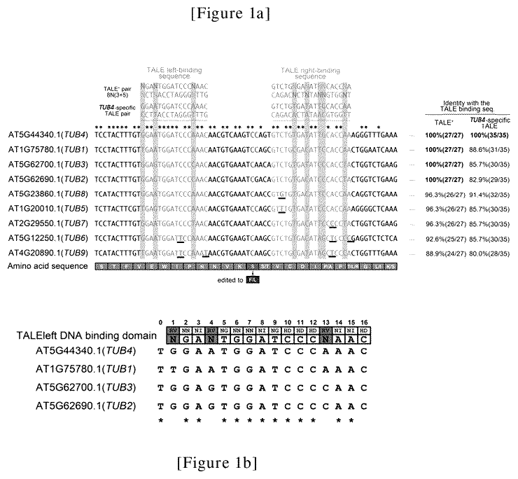

It has been known that, in the β-tubulin gene tub4 in the Arabidopsis thaliana genome, a phenotype of superficial cell rows and twisted primary roots is generated by a nucleotide substitution causing Ser351Phe (Ishida et al., Proceedings of the National Academy of Sciences, 104:8544-8549, 2007). This serine residue is conserved in all of the 9 β-tubulin genes present in the Arabidopsis thaliana genome, and it was aimed to simultaneously introduce a nucleotide substitution mutation into these corresponding serine residues as targets. There was designed a 15-base-long target window (a sequence flanked by the recognition sequences of TALE left and TALE right), comprising a target cytosine nucleotide that causes a mutation from Ser to Phe or from Ser to Leu, to this serine residue, by a nucleotide substitution from cytosine to thymine (FIG. 1a).

The TALECD used in this experiment was produced by modifying the platinum TALEN scaffold (Nakazato et al., Nature Plants 7:906-913, 2021), and this scaffold tends to have a higher affinity for the recognition sequence when the nucleotide adjacent to the 5′ of the recognition sequence of the TALE DNA-binding domain is thymine (Miller et al., Nature Biotechnology, 29:143-148, 2011). The recognition sequences each consisting of 16 and 19 nucleotides were set on the left and the right, so that thymine could be adjacent to the 5′ of the recognition sequence, and there were designed the left and right TALE binding domains with repeat sequences corresponding to the nucleotides that constitute the recognition sequence (FIG. 1a and b).

1-3. Production of nTALECD Expression Construct

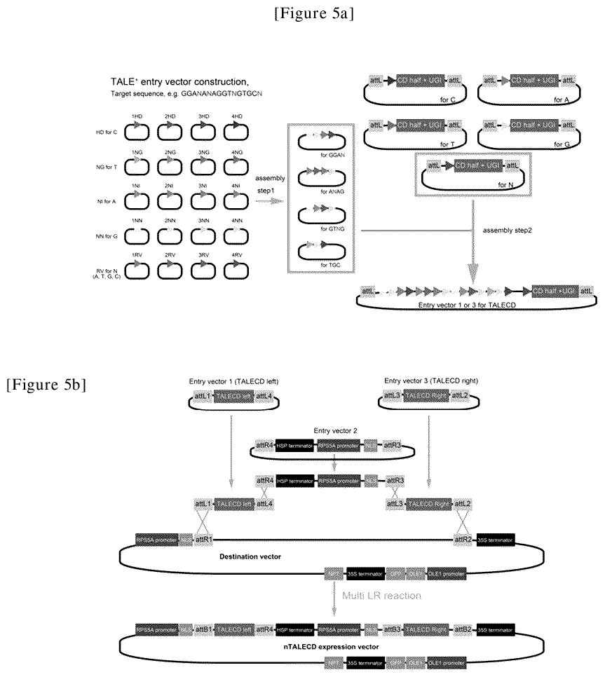

Using the Platinum Gate TALEN kit (Addgene, ID: #1000000043, Sakuma et al., Scientific Reports, 3:3379, 2013), two-step cloning was performed on an assembly of the DNA-binding domains of nTALECD. In the first step, 16 types of Platinum Gate TALEN module plasmids and the RV module plasmids p1-4 prepared in the previous section were combined, and were then subjected to a ligation reaction with array plasmids in the presence of Bsal-HFv2 (NEB), so that plasmids having 4 consecutive RVD repeat sequences in any given combination were cloned (FIG. 5a, left). In the second step, the multiple array plasmids produced in the first step were subjected to a ligation reaction in the presence of Esp3I (Thermo Fisher), so that the array plasmids were incorporated into entry vectors (e.g., E1_pENTR_L1-L4_NI_G1397-DddtoxA-N/#171727 and pENTR_E1_pF5A_L1-L4/#158728) each having the coding sequence of a protein (or Fok I nuclease, etc.), in which the RVD repeat of the C-terminus of the DNA-binding domain, the N-terminus or C-terminus of cytidine deaminase and uracil glycosylase inhibitor were ligated (FIG. 5a right).

The entry vectors (FIG. 5b, Entry vector 1 and Entry vector 3) having the full-length coding sequences of the left and right TALECD proteins were mixed with the entry vector (Entry vector 2) having an Arabidopsis thaliana RPS5A promoter, a nuclear localization signal (SV40NLS) and an HSP terminator sequence, a destination vector, and LR Clonase II Plus enzyme (Thermo Fisher), so as to produce a binary vector expressing the left and right TALECD proteins in tandem, using a multisite Gateway LR reaction (Thermo Fisher) (FIG. 5b).

### 1-4. Transformation and Screening of Transformants

A binary vector having an nTALECD expression cassette was introduced into the Agrobacterium strain C58C1 (pMP90) by electroporation. Arabidopsis thaliana wild-type Col-0 was transformed by infecting it with the Agrobacterium, into which the binary vector had been introduced, by an inflorescence immersion method (Clough and Bent, The Plant Journal, 16:735-743. 1998). Since the binary vector used for transformation has an expression cassette of Ole1 promoter: : Ole1-GFP, which is specifically expressed in seeds, the transformed seeds by this binary vector emit GFP fluorescence (Shimada et al., The Plant Journal, 61:519-528, 2010). Among the self-pollinated progeny seeds of individual plants infected with Agrobacterium, seeds exhibiting GFP fluorescence were seeded on 1/2 MS medium containing 125 μg/mL Claforan and 100 mg/mL sucrose, and the resulting T₁ seedlings were then used for analysis.

### 1-5. Growth Conditions and Genotyping

T₁ seeds were treated at low temperature of 4° C., were then transferred to an artificial climate chamber, and were then allowed to grow at 22° C. under long-day conditions (16-hour light period/8-hour dark period). Total DNA was extracted from one true leaf of the seedlings 14 days after the low-temperature treatment of the seeds. Using this total DNA as a template, PCR Sanger sequencing was carried out, and the sequence waveform data of the target sequence was analyzed on Geneious Prime (v. 2022. 1.1), and genotyping was performed for the target nucleotides. The primers used for amplification of PCR amplicons and Sanger sequencing are shown in Table 2.

TABLE 2

| Primer Name | Primer Sequence (5' to 3') | Purpose |
|---|---|---|
| TUB1-4_Fw | GGAGCAAAGTTCTGGGAAG (SEQ ID No: 43) | Amplification |
| TUB1-4_Rv | CTGAACATAGCTGTGAACTG (SEQ ID No: 44) | of TUB1~4 |
| TUB1_int2_Fw | CCTAGCTGTAAGTACCATC (SEQ ID No: 45) | Seq of TUB1 |
| TUB2_int2_Fw | GTTTGTTGAGTTTACTGTCTG (SEQ ID No: 46) | Seq of TUB2 |
| TUB3_int2_Fw | GTTTCTGCAAGATATGTTG (SEQ ID No: 47) | Seq of TUB3 |
| TUB4_int2_Fw | TTGTGTATTCATGTAGTGTG (SEQ ID No: 48) | Seq of TUB4 |
| TUB5-9_Fw | CGTCTCCACTTCTTCATGGT (SEQ ID No: 49) | Amplification |
| TUB5-9_Rv | CCTTCACCTGTGTACCAATG (SEQ ID No: 50) | of TUB5~9 |
| TUB5 ex3_Fw | CTCGCAACAATACATCTCA (SEQ ID No: 51) | Seq of TUB5 |
| TUB6 ex3 Fw | GTCTCAGCAGTACCGTGCA (SEQ ID No: 52) | Seq of TUB6 |
| TUB7_ex3_Fw | GATCTCAGCAGTACCGTAA (SEQ ID No: 53) | Seq of TUB7 |
| TUB8_ex3_Fw | ATCCACGCCACGGCAGG (SEQ ID) No: 54) | Seq of TUB8 |
| TUB9_ex3_Fw | AGCTGATCCTCGTCATGGT (SEQ ID) No: 55) | Seq of TUB9 |

### 2. Results

### 2-1. Design of TALEs That Simultaneously Recognize or Tolerate Gene Sequences Having Multiple SNPs

In a series of experiments disclosed in the present description, in actual plant bodies, an attempt was made to allow a repeat sequence having RVD that is not usually used, to recognize multiple different nucleotides. Moreover, with regard to, what is called, multigenes that are classified into the same gene family but have slightly different nucleotide sequences, a multiple-nucleotide-recognizing RVD repeat was used to tolerate SNPs thereof, and whether or not single-nucleotide mutations by genome editing could be simultaneously introduced into multiple loci (loci with slightly different nucleotide sequences) was verified.

Using nTALECD, a mutation to Phe or Leu was attempted to be caused to Ser351 of TUB2, TUB3, TUB4, TUB6, TUB7, TUB8, and TUB9, and Ser352 of TUB1 and TUB5, which are conserved in all of the 9 β-tubulin genes present in the Arabidopsis thaliana genome (FIG. 1a). The right view of FIG. 1a shows how many of the sequences of individual genes are recognized without mismatches (provided that the nucleotides recognized by N-recognizing RVD repeats in TALE+ are excluded), among the nucleotides of the target sequences recognized by individual TALEs (TALE+ and TUB4-specific TALE).

Taking the configuration of the repeat sequence of the TALE left of TALE+ 8N as an example, the design of the DNA-binding domain that simultaneously recognizes sequences having multiple SNPs is explained (FIG. 1b). Among the TALE recognition sequences (16 bases long to which the repeat sequence binds +1 nucleotide adjacent to the 5′) of the targets TUB1, TUB2, TUB3 and TUB4, the configurations of the 1st, 4th, and 13th nucleotides are different among genes. The DNA-binding domain of the TALE left was designed to recognize these three sites, using a repeat comprising RVD called RV that is not generally used.

2-2. Analysis of T₁ Individuals With Mutations Introduced

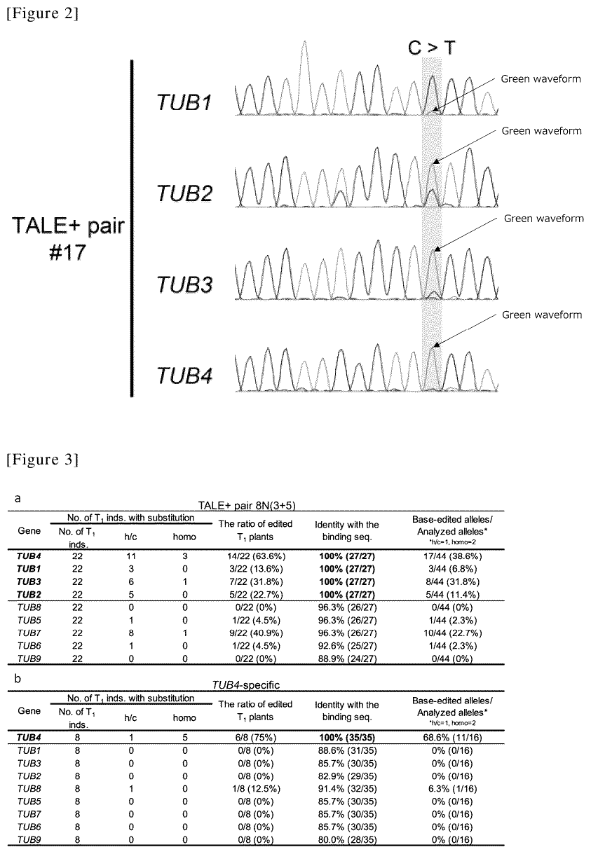

Regarding the first generation of transformants (T₁ generation), in which the expression vector of nTALECD had been introduced into the nuclear genome, PCR Sanger sequencing was used to confirm whether mutations were introduced into the target window 14 days after the low temperature treatment of the seeds. FIG. 2 shows the Sanger sequencing waveform of a representative individual (#17), which shows that partial or complete (homo) nucleotide substitutions (C>T) to the target nucleotide occurred at the four target loci of TUB1, TUB2, TUB3 and TUB4.

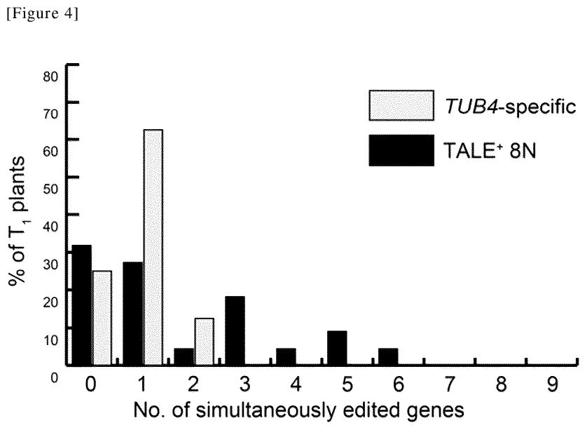

In the T₁ generation, into which TALE+ 8N pair or TUB4-specific pair constructs had been introduced, the number and percentage of individuals, in which mutations had been introduced into the target nucleotide of each β-tubulin gene, were summarized (FIG. 3). Mutations were introduced into the target TUB4 with high efficiency by the TUB4-specific pair, whereas no mutations were detected in the other eight β-tubulin genes, except for one individual in which a mutation was introduced into TUB8 (FIG. 3b). In contrast, in the case of the TALE+ 8N pair, mutations were introduced into the 4 targeted β-tubulin genes in multiple individuals, and among the 5 non-targeted β-tubulin genes, mutations were also introduced into TUB5, TUB6 and TUB7 in some individuals (FIG. 3a).

Next, the number of mutated β-tubulin genes in each T₁ individual was examined, and the percentage of the number of individuals to the number of edited genes was summarized (FIG. 4). The number of genes in which mutations were introduced by the TUB4-specific pair was 1 for TUB4 alone, and 2 for TUB4 and TUB8, whereas the number of genes by the TALE+ 8N pair varied from 1 gene to 6 genes.

From the results shown in the above FIG. 2 to FIG. 4, it was demonstrated that a single TALECD pair using the N-recognition RVD repeat can tolerate SNPs in the recognition sequence and can simultaneously target multiple similar sequences, compared to the case of using only the conventional each nucleotide-specific RVD repeat.

### INDUSTRIAL APPLICABILITY

By using the method or the protein according to the present invention, simultaneous modification of multiple genes becomes possible. Therefore, it is expected to utilize the method or the protein according to the present invention in the medical field, the agricultural field, and the livestock field.

1. A method for modifying multiple DNAs encoding identical or similar proteins, comprising:
allowing the TALE (transcription activator-like effector) portion of one type of TALE-modifier complex comprising at least one repeat sequence containing RVD (repeat variable di-residue) composed of amino acids that recognize or tolerate N, V, H, D, B, R, Y, M, W, S or K, to bind to the binding regions of the multiple DNAs.

2. A method for modifying multiple genes encoding identical or similar proteins in a cell, comprising:
introducing one type of TALE-modifier complex comprising at least one repeat sequence containing RVD composed of amino acids that recognize or tolerate N, V, H, D, B, R, Y, M, W, S or K, into the cell.

3. A method for producing a cell, in which multiple genes encoding identical or similar proteins in the cell are modified, comprising:
introducing one type of TALE-modifier complex comprising at least one repeat sequence containing RVD composed of amino acids that recognize or tolerate N, V, H, D, B, R, Y, M, W, S or K, into the cell.

4. The method according to claim 1, wherein the nucleotide(s) recognized or tolerated by the RVD are nucleotide(s), in which when the nucleotide sequences of the multiple DNAs or genes are aligned, one or multiple nucleotides present at the same positions are different from the nucleotides of other DNAs or genes.

5. The method according to claim 1,
wherein the amino acids of the RVD (i) are composed of RV, CS, VR, NA, S*, RH, RL or RT that recognize or tolerate N, (ii) are composed of HC or KC that recognize M, (iii) are composed of HS, HT, HV, KV or RC that recognize V, or (iv) are composed of NT that recognize R or V, and
wherein the “*” in S* indicates that the second digit of the RVD is a gap.

6. The method according to claim 1, wherein the modifier is whole or a part of an endonuclease, or whole or a part of a deaminase.

7. The method according to claim 2, wherein the genes are nuclear genes, mitochondrial genes or plastid genes.

8. The method according to claim 2, wherein the cell is a plant cell.

9. The method according to claim 3, wherein the cell is a plant cell.

10. A plant cell produced by the method according to claim 9.

11. A seed or a plant, comprising the plant cell according to claim 10.

12. A DNA-binding protein, comprising:
at least one repeat sequence of TALE,
wherein the RVD comprised in the repeat sequence (i) is composed of RV, CS, VR, NA, S*, RH, RL or RT that recognize or tolerate N, (ii) is composed of HC or KC that recognize M, (iii) is composed of HS, HT, HV, KV or RC that recognize V, or (iv) is composed of NT that recognize R or V, and
wherein the “*” in S* indicates that the second digit of the RVD is a gap.

13. The protein according to claim 12, wherein the RVD is RV that recognize or tolerate N.

14. The protein according to claim 13, wherein the protein fuses with a functional protein.

15. The method according to claim 2, wherein the nucleotide(s) recognized or tolerated by the RVD are nucleotide(s), in which when the nucleotide sequences of the multiple DNAs or genes are aligned, one or multiple nucleotides present at the same positions are different from the nucleotides of other DNAs or genes.

16. The method according to claim 3, wherein the nucleotide(s) recognized or tolerated by the RVD are nucleotide(s), in which when the nucleotide sequences of the multiple DNAs or genes are aligned, one or multiple nucleotides present at the same positions are different from the nucleotides of other DNAs or genes.

17. The method according to claim 2,
wherein the amino acids of the RVD are (i) composed of RV, CS, VR, NA, S*, RH, RL or RT that recognize or tolerate N, (ii) are composed of HC or KC that recognize M, (iii) are composed of HS, HT, HV, KV or RC that recognize V, or (iv) are composed of NT that recognize R or V, and
wherein the “*” in S* indicates that the second digit of the RVD is a gap.

18. The method according to claim 3,
wherein the amino acids of the RVD (i) are composed of RV, CS, VR, NA, S*, RH, RL or RT that recognize or tolerate N, (ii) are composed of HC or KC that recognize M, (iii) are composed of HS, HT, HV, KV or RC that recognize V, or (iv) are composed of NT that recognize R or V, and
wherein the “*” in S* indicates that the second digit of the RVD is a gap.

19. The method according to claim 3, wherein the modifier is whole or a part of an endonuclease, or whole or a part of a deaminase.

20. The method according to claim 3, wherein the genes are nuclear genes, mitochondrial genes or plastid genes.
